(19) United States
(12) Patent Application Publication
(10) Pub. No.: US 2026/0053361 A1
(43) Pub. Date: Feb. 26, 2026

(54) AI-ENABLED WIRELESS WEARABLE MEDICAL DEVICE FOR CONTINUOUS AUSCULTATION, VITAL SIGN MONITORING, AND ANALYSIS

(71) Applicant: ResperCare, LLC, Pittsboro, IN (US)

(72) Inventors: Todd C. Bracken, Pittsboro, IN (US); Sheri A. Sherman, Pittsboro, IN (US); Andrew Kaeleb Sherman, Linton, IN (US); Tommaso Calviello, Terre Haute, IN (US); Connor Shea O'Connell, Fort Myers, FL (US); Alyssa Jane McGauley, Morgantown, IN (US); Kathryn Anne Choi, Lake Zurich, IL (US); Amanda Noelle Kuhlman, Noblesville, IN (US)

(21) Appl. No.: 19/305,115

(22) Filed: Aug. 20, 2025

Related U.S. Application Data
(60) Provisional application No. 63/685,387, filed on Aug. 21, 2024.

(51) Int. Cl. A61B 5/00; A61B 5/01; A61B 5/024; A61B 5/1455; A61B 7/04

(52) U.S. Cl. CPC A61B 5/0024; A61B 5/0008; A61B 5/015; A61B 5/02427; A61B 5/14552; A61B 5/6833; A61B 5/684; A61B 5/7225; A61B 5/7445; A61B 5/746; A61B 7/04; A61B 2560/0462; A61B 2562/0271

(57) ABSTRACT

An electronic stethoscope assembly comprises a plurality of patches and a controller. The plurality of patches include at least one sensor configured to measure physiological signals upon placement in contact with the patient and a transceiver configured to transmit physiological signal data. The controller is in communication with at least one of the plurality of patches to receive a set of physiological signal data therefrom for processing and analyzing of the set of physiological signal data so that the set of physiological signal data can be continuously collected and monitored to identify pathologies present in the set of physiological signal data.

### CROSS-REFERENCE TO RELATED APPLICATION

This patent application claims the benefit of, and priority to, U.S. Provisional Patent Application Ser. No. 63/685,387, filed 21 Aug. 2024, the entire disclosure of which is hereby incorporated herein by reference.

### TECHNICAL FIELD

The present disclosure relates generally to biomedical monitoring and diagnostic devices, and more specifically to a wireless, AI-enabled, wearable medical device configured for continuous auscultation, vital sign monitoring, and physiological signal analysis. The disclosed system integrates a disposable patch incorporating acoustic and physiological sensors with wireless transmission and AI-based telemetry processing, enabling real-time remote patient monitoring in clinical, post-acute, home care, and military battlefield settings.

### BACKGROUND

Traditional auscultation techniques rely on handheld mechanical or electronic stethoscopes, which require the physical presence of a caregiver to listen to internal body sounds. These approaches are episodic, manually operated, and provide only limited, point-in-time physiological data. While electronic stethoscopes may enable some degree of wireless amplification, they still require active patient engagement and caregiver manipulation, often disrupting sleep, mobility, or recovery, especially in vulnerable populations such as neonates or post-operative patients.

Continuous vital sign monitoring is typically achieved using multiple separate sensors, wires, and bedside monitors, which can be cumbersome, prone to motion artifacts, and unsuitable for ambulatory or home care environments. Existing systems often lack the ability to collect and interpret auscultatory sounds—such as breath, heart, or gastrointestinal sounds—in real time, and most do not combine acoustic and biometric data into a unified wearable platform.

Moreover, reusable hardware and tethered systems pose infection control challenges in sterile settings like emergency rooms (ERs), intensive care units (ICUs), critical care units (CCUs), and neonatal intensive care units (NICUs), where disposability, sterility, and skin safety are paramount. Devices that require recharging, frequent repositioning, or clinician-dependent data acquisition introduce additional operational burdens.

As the demand increases for decentralized care models—including hospital-at-home, early discharge monitoring, and chronic disease management—there is a need for a wearable, disposable, AI-enabled device capable of continuously capturing, transmitting, and interpreting a range of physiological signals. Such a device would improve early detection of clinical deterioration, reduce caregiver workload, and enable more timely and informed interventions.

Accordingly, there remains a need for an improved system that enables wireless, autonomous, and continuous auscultation and core vital sign monitoring from a single, compact medical patch. The present disclosure addresses these needs. Generally, traditional stethoscopes include mechanical means for listening to body sounds. For example, traditional stethoscopes include a handheld diaphragm connected to tubing and an earpiece for listening to body sounds. Some electronic stethoscopes may allow for wireless amplification of body sounds. However, these electronic stethoscopes are incapable of constant monitoring of the body sounds. Electronic stethoscopes require a caregiver to be present throughout the listening process. Moreover, these electronic stethoscopes require the patient to move to particular body positions to provide the caregiver access to various listening locations. The presence of the caregiver and the necessary movement of the patient may interrupt the patient's activities, such as sleep, which may impede healing. As such, improved systems and methods for monitoring and analyzing physiological signals would be advantageous.

### BRIEF DESCRIPTION OF THE DRAWINGS

FIG. 1 depicts an electronic stethoscope assembly for monitoring and analyzing physiological signals of a patient, the electronic stethoscope assembly including a plurality of patches, a network communicatively coupled to the plurality of patches, and an assembly controller communicatively coupled to the network;

FIG. 2 depicts a user interface included in the assembly controller;

FIG. 3 depicts an exemplary patch included in the plurality of patches;

FIG. 4 depicts a process for using the electronic stethoscope assembly;

FIG. 5 depicts a flow diagram of the physiological signals through the electronic stethoscope assembly;

FIG. 6 depicts a command patch and a follower patch included in the plurality of patches;

FIG. 7A depicts an alternative embodiment of a command patch;

FIG. 7B depicts the command patch of FIG. 7A;

FIG. 8 depicts exemplary positioning of the plurality of patches to monitor respiratory sounds;

FIG. 9 depicts exemplary positioning of the plurality of patches to monitor cardiovascular sounds;

FIG. 10 depicts exemplary positioning of the plurality of patches to monitor gastrointestinal sounds; and

FIG. 11 depicts exemplary positioning of the plurality of patches to monitor temperature and/or oxygen saturation.

### DETAILED DESCRIPTION OF THE DRAWINGS

For the purposes of promoting an understanding of the principles of the disclosure, reference will now be made to a number of illustrative embodiments illustrated in the drawings and specific language will be used to describe the same.

The present disclosure provides an electronic stethoscope assembly 10 for monitoring and analyzing physiological signals of a patient. The electronic stethoscope assembly 10 allows for constant monitoring and analysis of the physiological signals without the physical presence of a caregiver and without requiring the patient to move to particular body positions to obtain the physiological signals.

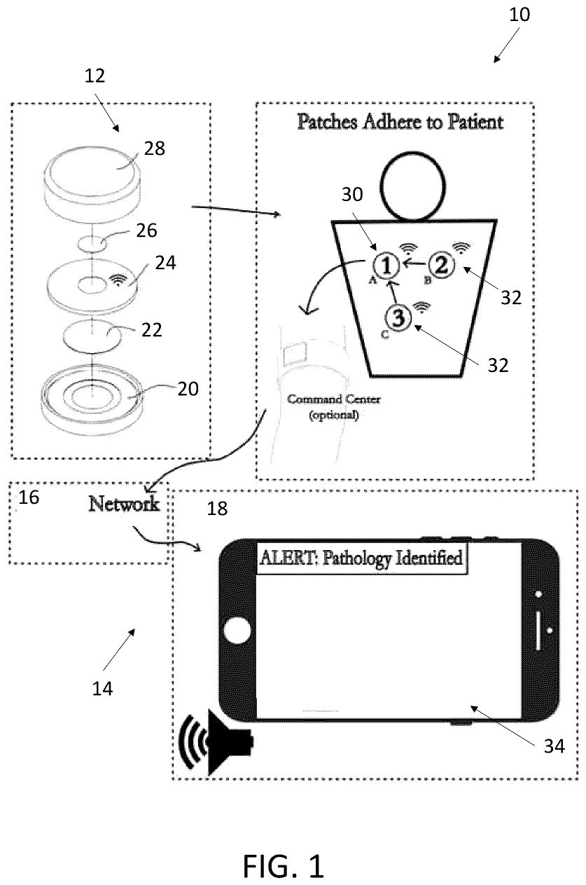

The electronic stethoscope assembly 10 includes a plurality of patches 12 and a control system 14, as shown in FIG. 1. Each patch included in the plurality of patches 12 is positioned and/or adhered to the patient at a desired observation location on a body of the patient. The control system 14 is in communication with the plurality of patches 12 to receive physiological signal data therefrom. The control system 14 illustratively includes a network 16 and an assembly controller 18, as shown in FIG. 1. In use, each patch included in the plurality of patches 12 senses and/or detects respective physiological signals, and physiological signal data as detected by each patch is transmitted to the network 16. The network 16 then transmits the physiological signal data as detected by each patch to the assembly controller 18 for use and viewing by the patient and/or the caregiver.

One or more patches 12 may be used for the detection of any particular physiological signal corresponding to at least one physiological function or operation of the patient. Physiological signals are produced by the body while the systems of the body function, and the physiological signals reflect the state of those systems. The physiological signals may be internal or may emanate from the body. The physiological signals that may be detected, analyzed, and monitored include physiological signals related to, but not limited to, respiratory sounds, cardiovascular sounds, gastrointestinal sounds, body temperature, oxygen saturation, blood pressure, heart rate, among others. The desired observation location of each patch included in the plurality of patches 12 on the body of the patient is dependent on the desired physiological signals for monitoring. One or more patches 12 may be used for the detection of one physiological signal, and another one or more patches 12 may be used for the detection of a different physiological signal.

The electronic stethoscope assembly 10 may be used in hospitals, care facilities, physician offices, or at home, among other settings. For example, in some embodiments, the electronic stethoscope assembly 10 is integrated with hospital software using, for example, Cerner, or other software platforms.

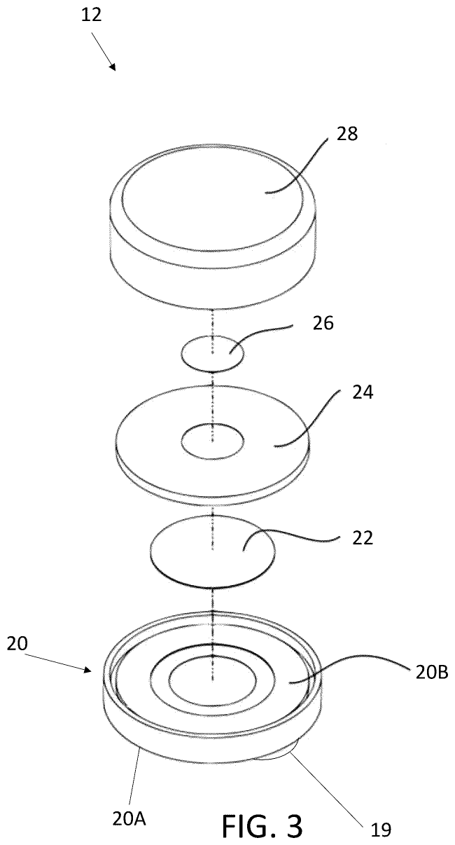

In illustrative embodiments, each patch included in the plurality of patches 12 includes an adhesive surface configured to be adhered to skin of the patient at the desired observation location. For example, each patch included in the plurality of patches 12 includes a base 20 having an adhesive layer (not shown) on an underside 20A of the base 20, and a peelable layer 19 covering the adhesive layer, as shown partially peeled away from the underside 20A in FIG. 3. The peelable layer 19 protects the surface of the underside 20A of the base before use of the plurality of patches 12. The peelable layer 19 is peeled off each of the plurality of patches 12 to expose the adhesive layer on the underside 20A of the base 20. The underside 20A of the base 20 is the mounting surface of the base 20, and of the patch 12 generally, and the base 20 of each of the plurality of patches 12 is adhered to the patient via the adhesive layer on the underside 20A, i.e., mounting surface, of the base 20. The patient and/or the caregiver adheres each of the plurality of patches 12 to the desired observation location. The patient and/or the caregiver set the desired observation locations.

Each patch included in the plurality of patches 12 may be preset to a specific RFID channel, or other wireless communication channel, for communication between the plurality of patches 12. Each patch included in the plurality of patches 12 establishes a connection to the other patches included in the plurality of patches 12. In some embodiments, the plurality of patches 12 is configured to connect automatically to a receiver near the patient or to the Wi-Fi network 16. The Wi-Fi network 16 may be the Wi-Fi network at the hospital, for example, or the Wi-Fi network at the patient's home, as another example.

Each patch included in the plurality of patches 12 includes at least one sensor 22, a printed circuit board 24, a battery 26, and a protective cap 28, all mounted to the top side 20B of the base 20 as shown in FIG. 3. For simplicity sake, the components of a single patch included in the plurality of patches 12 is described below. However, each patch included in the plurality of patches 12 is illustratively identical such that the description applies to each patch included in the plurality of patches 12.

The at least one sensor 22 is located adjacent to the base 20 of the patch 12 so that the at least one sensor 22 is in close proximity to the skin of the patient. The at least one sensor 22 fits into the base 20, as suggested in FIG. 3. The at least one sensor 22 is attached to the printed circuit board 24 to locate the at least one sensor 22 between the base 20 and the printed circuit board 24. The battery 26 powers the at least one sensor 22 and the printed circuit board 24. The battery 26 is coupled to the printed circuit board 24, as suggested in FIG. 3. The protective cap 28 and the base 20 cooperate to secure the least one sensor 22, the printed circuit board 24, and the battery 26 therebetween. In some embodiments, the protective cap 28 comprises noise-attenuating materials. In some embodiments, the protective cap 28 includes a serial number to identify the individual patch 12.

In some embodiments, the at least one sensor 22 is or includes a transducer configured to detect acoustic sound waves. In some embodiments, the at least one sensor 22 is or includes a microphone. In some embodiments, the at least one sensor 22 is or includes a contact microphone. In some embodiments, the at least one sensor 22 is or includes a temperature sensor configured to detect a body temperature of the patient. In some embodiments, the at least one sensor 22 is or includes a photoplethysmogram (PPG) sensor configured to detect a blood oxygen saturation of the patient and/or a heart rate of the patient.

In some embodiments, one patch 12 includes a single sensor 22 therein, however, each patch included in the plurality of patches 12 may contain a different sensor 22 therein as compared to the other patches 12 included in the plurality of patches 12. In some embodiments, one patch 12 includes the transducer, the temperature sensor, and the PPG sensor. In some embodiments, the patch 12 includes at least one acoustic transducer and/or microphone, at least one temperature sensor, at least one PPG sensor, and/or any other one or more sensors, or any combination of sensors. Thus, the patch 12 may contain fewer than, more than, or a different set of sensors 22 than the sensors 22 described herein.

The printed circuit board 24 includes filtering circuitry, amplifying circuitry, a microcontroller, a receiver, and/or a transmitter. In some embodiments, a conventional transceiver may be used instead of separate receivers and transmitters. Physiological signals are output from the at least one sensor 22 to the filtering circuitry and/or the amplifying circuitry. The amplifying circuitry amplifies the physiological signals, as necessary. The filtering circuitry may attenuate one or more frequency bands of the physiological signals, as necessary. The band of attenuation may be modified based on the desired physiological signal for monitoring. The patient and/or the caregiver may, in some embodiments, set the desired band(s) of attenuation. Filtered physiological signal data is digitized and organized using the microcontroller.

In some embodiments, the patch included in the plurality of patches 12 with the fastest connection to the network 16 is set as a command patch 30 (i.e., the first patch to connect to the network 16). The remaining patches included in the plurality of patches 12 are follower patches 32. The command patch 30 is configured to transmit, process, and receive physiological signal data from the follower patches 32. Illustratively, the plurality of patches 12 includes at least one command patch 30 and at least one follower patch 32.

The follower patches 32 are communicatively coupled with the command patch 30. Each of the follower patches 32 transmits the respective physiological signal data to the receiver of the command patch 30 via the transmitter included in each of the follower patches 32. The command patch 30 receives the physiological signal data from each of the follower patches 32 and a tag associated with the physiological signal data from each of the follower patches 32 so that the data is associated with a particular follower patch 32. The command patch 30 transmits the received physiological signal data to the network 16 via the transmitter of the command patch 30.

The physiological signal data received by the command patch 30 may include phase-encoding information or may originate from a multi-directional antenna. In some embodiments, the command patch 30 uses the physiological signal data from the follower patches 32 to determine the physical arrangement of the follower patches 32 around the body of the patient. The physical arrangement of the follower patches 32 on the patient, as determined by the command patch 30, is transmitted to the network 16 by the command patch 30.

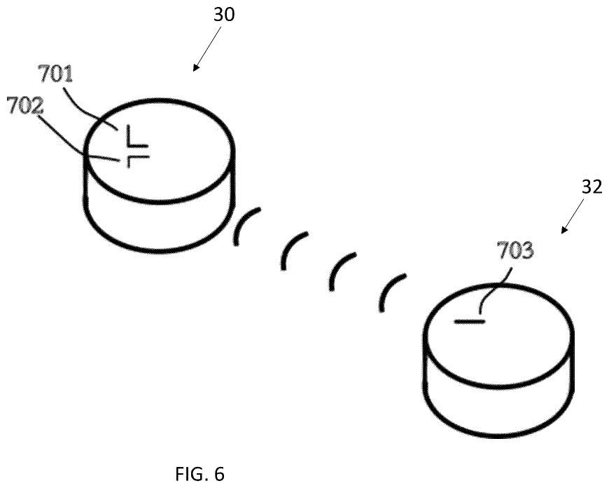

To determine the physical arrangement of the follower patches 32 on the body of the patient, in some embodiments, the command patch 30 uses an angle of radio frequency arrival, as shown in FIG. 6. The follower patches 32 transmit the physiological signal data using a unidirectional antenna 703. At the command patch 30, one antenna 701 can determine the vertical direction of arrival of the physiological signal data. Another antenna 702 can determine the horizontal direction of arrival of the physiological signal data. The strength of the physiological signal data after attenuation can be used to determine the distance to the respective follower patch 32. Combined, this information provides the position of each follower patch 32 relative to the command patch 30, which is transmitted to the network 16 from the command patch 30.

The network 16 allows the physiological signal data to be recorded, uploaded, and stored in a memory, such as a cloud. From the network 16, the physiological signal data is transmitted to the assembly controller 18, as suggested in FIG. 1. The assembly controller 18 may illustratively be a computer, a tablet, or a mobile phone. The assembly controller 18 includes a user interface 34. Caregivers and/or patients may view the physiological signal data in real time on the user interface 34.

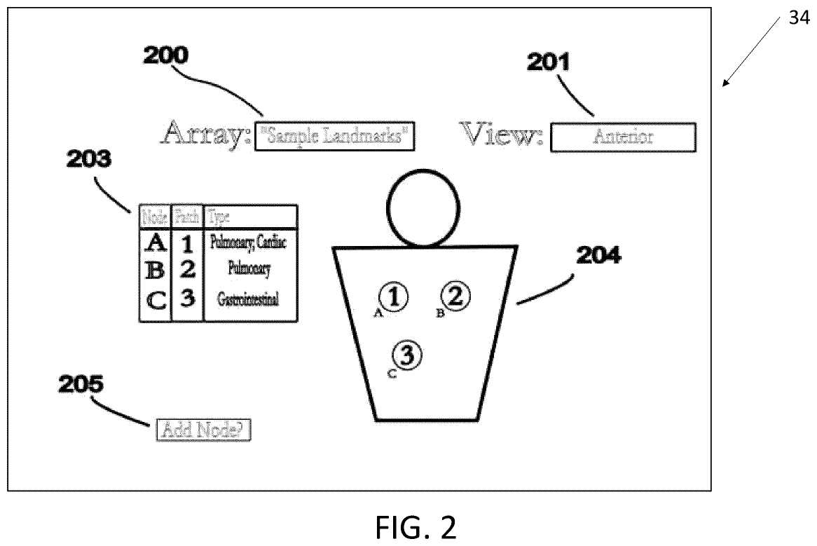

Caregivers and/or patients set the desired observation location of the plurality of patches 12 using the user interface 34. For example, the caregiver and/or the patient can select the type of array 200 to be used (i.e., the desired observation location of the plurality of patches 12 on the patient for the physiological signal of interest). In some embodiments, the user interface 34 shows a map 204 of the body of the patient and a labeling of the plurality of patches 12 on the patient based on the array 200 selected, as shown in FIG. 2. The map 204 may be viewed by the caregiver and/or the patient to aid in adhering the patches 12 to the patient. The caregiver and/or the patient may set the band(s) of attenuation using the user interface 34 for each patch included in the plurality of patches 12. In this way, the caregiver can alter the physiological signal being detected by each patch. Depending on the desired physiological signal, the frequency-attenuation characteristics of each patch 12 will be adjusted.

The desired band of attenuation of each patch is transmitted to the network 16 from the assembly controller 18. The network 16 then transmits the desired band(s) of attenuation to the command patch 30, which transmits the desired band(s) of attenuation to the receiver of each of the follower patches 32. Each follower patch 32 may have a different desired band or bands of attenuation.

While in use, in some embodiments, the user interface 34 includes visualization features, such as spectrograms. Patches 12 with detected pathologies may be highlighted or indicated in the map 204. The map 204 will update to show the physical arrangement of the follower patches 32 on the patient, as determined by the command patch 30. A table 203 lists the patches 12 and the type of physiological signal each patch 12 is set to detect, as shown in FIG. 2. If the location of the patches 12 as determined by the command patch 30 is incorrect such that the map 204 is incorrect, the caregiver and/or the patient can modify the location of the incorrectly positioned patch 12 using the table 203. The serial number included on each patch 12 can aid the caregiver and/or the patient in correcting the location of the patch 12. The caregiver and/or the patient can add a new patch to the array 200 via a button 205. A view button 201 is used to switch the map 204 between an anterior and posterior view of the patient, as shown in FIG. 2.

The physiological signal data is analyzed in discrete time intervals by a processor of the network 16. The time interval may be any time interval, such as, for example, five seconds, 10 seconds, 15 seconds, etc. In some embodiments, the time interval is a minute, two minutes, three minutes, four minutes, etc. Processing techniques such as, for example, spectral analysis can be used to identify the presence of pathologies within the physiological signal data. AI processing systems may also be used to identify the presence of pathologies. When pathologies are detected, a notification or a warning may be sent to the caregiver and/or the patient. For example, alert notifications can be sent to the assembly controller 18 to call attention to the detected pathologies. The alert may be a visual alert, an auditory alert, or a combination of the same. Caregivers may add additional diagnostic information to the network 16 to supplement the database. Additional data can be used to train the AI model and improve the accuracy of the model.

The electronic stethoscope assembly 10 is customized to the patient as the caregiver designates the desired observation location for each patch 12. Additionally, the caregiver designates the physiological signal for detection at each patch 12.

Illustratively, the command patch 30 and the follower patches 32 are the same. If the command patch 30 fails, one of the follower patches 32 will be chosen as the command patch 30.

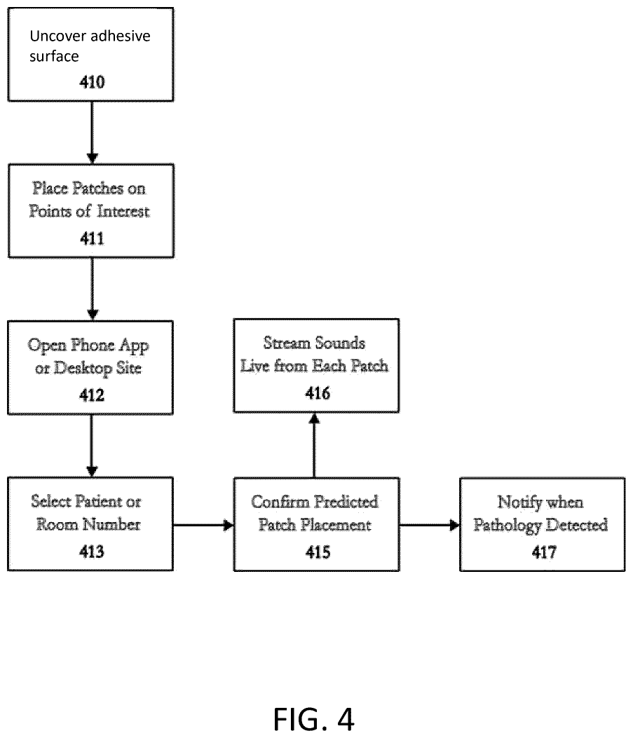

As shown in FIG. 4, a process for using the electronic stethoscope assembly 10 is depicted. The process includes step 410 of removing the peelable layer of the patches 12 that protects the adhesive surface such that the patches 12 are ready to be adhered to the skin of the patient. In step 411, the patches 12 are positioned and adhered to the patient at the desired observation locations. In step 412, the user interface 34 is accessed. In step 413, patient identification information or a room number is selected to view the specific information as related to the patient on the user interface 34. In step 415, the map 204 is viewed to confirm that the patches 12 are adhered to the desired observation locations. If a patch 12 is incorrectly shown on the map 204, the caregiver and/or the patient can modify the map 204. In step 416, the caregiver and/or the patient can view the physiological signal data in real time from each patch 12. The caregiver and/or the patient may select a specific patch or group of patches 12 to view. In step 417, the caregiver and/or the patient receive an alert if a pathology is detected.

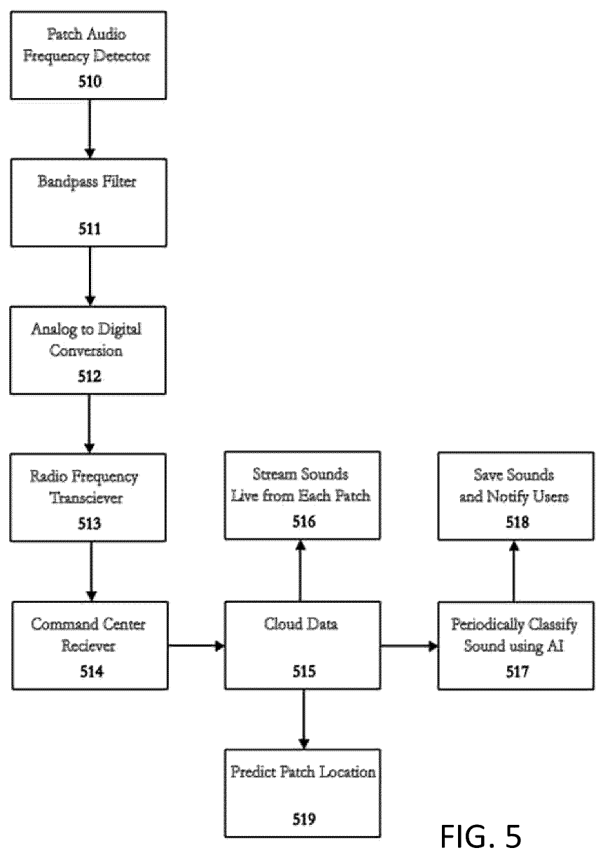

FIG. 5 depicts the flow of the physiological signals from the patches 12 to the user interface 34. In step 510, the physiological signals are collected using the at least one sensor 22. In step 511, a bandpass filter, accompanied by any necessary amplification, is applied to the physiological signal to ensure that only the physiological signal of interest is analyzed. In step 512, the physiological signals are converted to digital information. In step 513, the physiological signal data is transmitted, using radio frequency, to the command patch 30. In step 514, the command patch 30 transmits the physiological signal data from each follower patch 32 to the network 16. In step 515, the physiological signal data is saved in the memory of the network 16. In step 516, the physiological signal data can be streamed in real time from the network 16. In step 519, the location of each patch 12, as determined by the command patch 30, is stored in the memory of the network 16. In step 517, the physiological signal data is processed and/or analyzed by the processor of the network 16 at periodic intervals to identify pathologies. For example, the physiological signal data may be analyzed every 10 to 15 seconds. In step 518, if a pathology is detected, the physiological signal data related to that pathology is saved and tagged and an alert is sent to the caregiver and/or the patient.

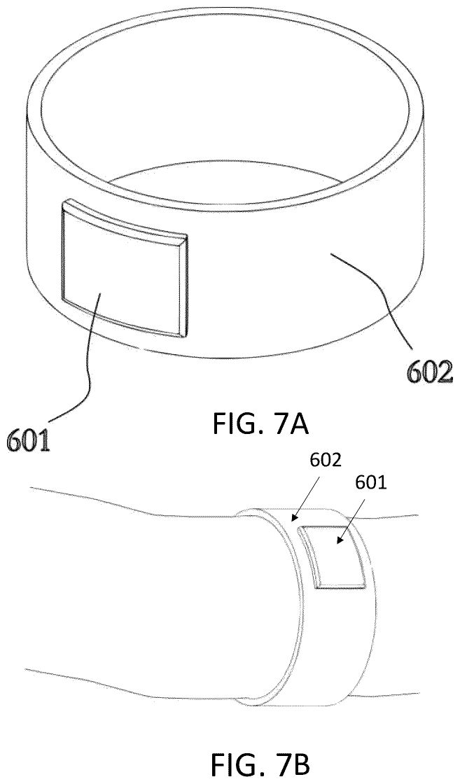

An alternative embodiment of a command patch is shown in FIGS. 7A and 7B. Illustratively, the command patch is a command center 601 that is not adhered to the skin of the patient. The command center 601 is located on a strap 602. The strap 602 may be a wristband, a necklace, or a ring, for example. The strap 602 may be formed of fabric, paper, rubber, plastic, or any other suitable alternatives. The command center 601 receives the physiological signal data from the follower patches 32 and transmits the physiological signal data to the network 16.

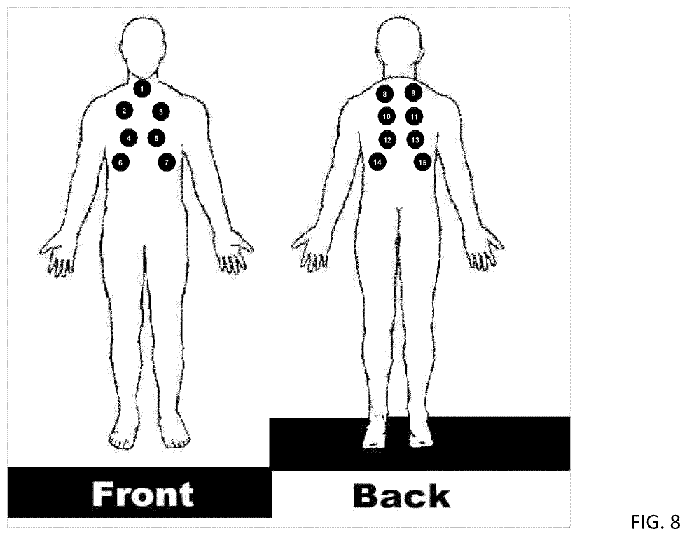

In some embodiments, at least one of the plurality of patches 12 is used to monitor respiratory sounds. The respiratory sounds may comprise vesicular sounds, bronchial sounds, bronchovesicular sounds, tracheal breath sounds, and/or respiration rate, among other respiratory sounds. The respiratory sound telemetry that is continuously captured includes, but is not limited to, a pitch of the sound, a frequency of the sound, a duration of the sound (i.e., how long the sound lasted), an amplitude of the sound, a height of the sound, an intensity of the sound, a timing of the sound (i.e., the time at which the sound occurred), a tone of the sound (i.e., a sound quality), a harmonic of the sound, and/or a respiratory rate, among others. To analyze specific respiratory sounds, particular observation locations for the plurality of patches 12 are chosen. Exemplary observation locations for respiratory sounds are shown in FIG. 8 for the front and the back of the patient. Exemplary patches 12 that may be used for each type of respiratory sound are shown in Table 1. It should be understood that Table 1 is merely exemplary and more patches may be used for a particular sound, less patches may be used for a particular sound, or different locations may be used for a particular sound.

TABLE 1

| Type of Respiratory | | |
|---|---|---|
| Sound | Front Patch Placement | Back Patch Placement |
| Vesicular Sounds | #2, #3, #4, #5, #6, #7 | #8, #9, #10, #11, #12, |
| | | #13, #14, #15 |
| Bronchial Sounds | #1, #2, #3, #4, #5, #6, | |
| | #7 | |
| Bronchovesicular | #2, #3, #4, #5, #6, #7 | #8, #9, #10, #11, #12, |
| Sounds | | #13, #14, #15 |
| Tracheal Breath Sounds | #1, #2, #3, #4, #5, #6, | |
| | #7 | |
| Respiration Rate | #2, #3, #4, #5, #6, #7 | #8, #9, #10, #11, #12, |
| | | #13, #14, #15 |

Pathologies or anomalies that may be monitored for and/or detected using respiratory sounds include, but are not limited to, stridor, rhonchi (i.e., low pitched wheezes), pulmonary edema, pleural function rub, fine crackles (i.e., rales, early inspiratory/late inspiratory), coarse crackles (i.e., rales, early inspiratory/late inspiratory), squawks, wheezes (inspiratory/expiratory, high pitched/low pitched, monophonic/polyphonic, pectoriloquy), pneumothorax (i.e., collapsed lung, absence of lung sound), egophony (e-a changes), pectoriloquy (normal/whispered), vesicular (normal/diminished), and/or sudden infant death syndrome, among others. Exemplary patches 12 that may be used for each pathology are shown in Table 2. It should be understood that Table 2 is merely exemplary and more patches may be used for a particular sound, less patches may be used for a particular sound, or different locations may be used for a particular sound.

TABLE 2

| Type of Pathology | Front Patch Placement | Back Patch Placement |
|---|---|---|
| Stridor | #1 | |
| (Frequencies: >500 Hz) | | |
| Rhonchi | #2, #3, #4, #5 | |
| (Frequencies: >150 Hz) | | |
| Pulmonary Edema | #2, #3, #4, #5, #6, #7 | #8, #9, #10, #11, #12, |
| | | #13, #14, #15 |
| Pleural Function Rub | #6, #7 | |
| (Frequencies: >350 Hz) | | |
| Fine Crackles | #6, #7 | #14, #15 |
| (Frequencies: >650 Hz) | | |
| Coarse Crackles | #6, #7 | #14, #15 |
| (Frequencies: >350 Hz) | | |
| Squawks | | #12, #13, #14, #15 |
| (Frequencies: 200-300, | | |
| 500 Hz) | | |
| Wheezes | #2, #3, #4, #5 | #10, #11, #12, #13 |
| Frequencies: 100-5000 | | |
| (400) Hz | | |
| Pneumothorax | #2, #3, #4, #5, #6, #7 | #8, #9, #10, #11, #12, |
| | | #13, #14, #15 |
| Egophony | #2, #3, #4, #5, #6, #7 | #8, #9, #10, #11, #12, |
| | | #13, #14, #15 |
| Pectoriloquy | #2, #3, #4, #5, #6, #7 | #8, #9, #10, #11, #12, |
| | | #13, #14, #15 |
| Vesicular | #2, #3, #4, #5, #6, #7 | #8, #9, #10, #11, #12, |
| | | #13, #14, #15 |
| Sudden Infant Death | #2, #3, #4, #5, #6, #7 | |
| Syndrome (SIDS) | | |

More than one type of respiratory sound and/or pathology may be monitored, analyzed, or detected at any one time.

In some embodiments, at least one of the plurality of patches 12 is used to monitor cardiovascular sounds. The cardiovascular sounds may comprise aortic sounds, pulmonic sounds, Erb's point sounds, tricuspid sounds, mitral sounds, first heart sounds (S1), second heart sounds (S2), third heart sounds (S3), fourth heart sounds (S4), heart rate/pulse rate sounds, general and peripheral vascular sounds, peripheral pulse sounds, apical pulse sounds, radial pulse sounds, carotid pulse sounds, jugular pulse sounds, and/or systolic and diastolic blood pressures sounds, among other cardiovascular sounds.

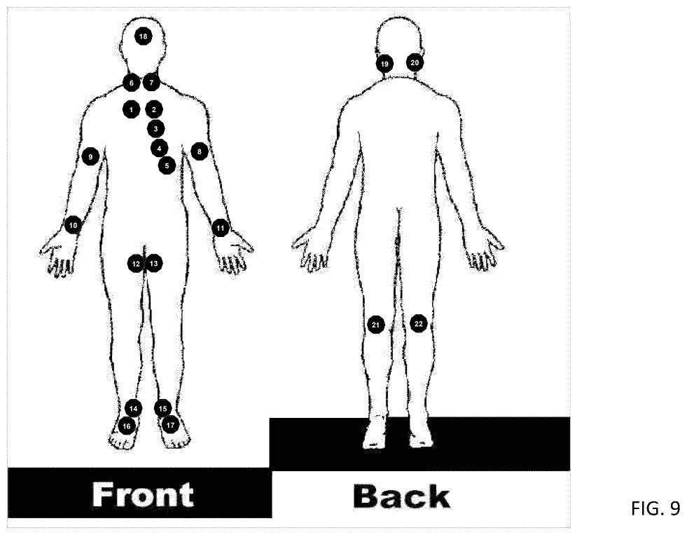

The cardiovascular sound telemetry that is continuously captured includes, but is not limited to, a pitch of the sound, a frequency of the sound, a duration of the sound (i.e., how long the sound lasted), an amplitude of the sound, a height of the sound, an intensity of the sound, a timing of the sound (i.e., the time at which the sound occurred), a tone of the sound (i.e., a sound quality), and/or a harmonic of the sound, among others. To analyze specific cardiovascular sounds, particular observation locations for the plurality of patches 12 are chosen. Exemplary observation locations for cardiovascular sounds are shown in FIG. 9 for the front and the back of the patient. Exemplary patches 12 that may be used for each type of cardiovascular sound are shown in Table 3. It should be understood that Table 3 is merely exemplary and more patches may be used for a particular sound, less patches may be used for a particular sound, or different locations may be used for a particular sound.

TABLE 3

| Type of Cardiovascular | | Back Patch |
|---|---|---|
| Sound | Front Patch Placement | Placement |
| Aortic Sounds | #1 | |
| Pulmonic Sounds | #2 | |
| Erb's Point Sounds | #3 | |
| Tricuspid Sounds | #4 | |
| Mitral Sounds | #5 | |
| Heart Rhythm Sounds | #1, #2, #3, #4, #5 | |
| First Heart Sounds (S1) | #1, #2, #3, #4, #5 | |
| Second Heart Sounds | #1, #2, #3, #4, #5 | |
| (S2) | | |
| Third Heart Sounds (S3) | #1, #2, #3, #4, #5 | |
| Fourth Heart Sounds (S4) | #1, #2, #3, #4, #5 | |
| Heart Rate/Pulse Rate | #1, #2, #3, #4, #5, #6, #7, | #21, #22 |
| Sounds | #8, #9, #10, #11, #12, | |
| | #13, #14, #15, #16, #17 | |
| General and Peripheral | #1, #2, #3, #4, #5, #6, #7, | #21, #22 |
| Vascular Sounds | #8, #9, #10, #11, #12, | |
| | #13, #14, #15, #16, #17 | |
| Peripheral Pulse Sounds | #1, #2, #3, #4, #5, #6, #7, | #21, #22 |
| | #8, #9, #10, #11, #12, | |
| | #13, #14, #15, #16, #17 | |
| Apical Pulse Sounds | #5 | |
| Radial Pulse Sounds | #10, #11 | |
| Carotid Pulse Sounds | #6, #7 | |
| Jugular Pulse Sounds | #6, #7 | |
| Systolic and Diastolic | #1, #2, #3, #4, #5, #6, #7, | #21, #22 |
| Blood Pressures Sounds | #8, #9, #10, #11, #12, | |
| | #13, #14, #15, #16, #17 | |

Pathologies or anomalies that may be monitored for and/or detected using cardiovascular sounds include, but are not limited to, arrhythmias, heart murmur, abnormal splitting, heart gallop, heart clicks, pericardial rub, muffled heart sounds, ascending aortic aneurysm, aortic stenosis, aortic, mitral, tricuspid valve regurgitation, carotid assessments, jugular vein distention assessments (JVD), ankle brachial index assessments (ABI), arteriovenous fistula assessments (AVF), arteriovenous graft assessments (AVG), aortic prosthetic heart sound aortic ejection click, aortic sclerosis (musical murmur, diamond shaped systolic murmur, mild, moderate, severe; regurgitation (rheumatic)), aortic stenosis, aortic regurgitation (mild, moderate, severe, decrescendo diastolic murmur), aortic coarctation, aortic ejection click—first heart sound plus aortic ejection click, aortic prosthetic heart sound, aortic intensity—second heart sound (fixed splitting, increasing), atrial septal defect, innocent murmur, patent ductus arteriosus, pulmonary stenosis, pulmonic regurgitation (mild, moderate, severe), second heart sound (fixed splitting, physiologic split, splitting, fixed splitting, decreased aortic intensity), acute pericarditis, mediastinal crunch, arrhythmogenic right ventricular dysplasia, Ebstein's anomaly, first heart sound (markedly split, minimally split), innocent systolic ejection murmur (standing, supine), tetralogy of fallot, tricuspid regurgitation (normal, moderate, severe), ventricular septal defect, commotio cordis, first heart sounds (normal, reduced intensity, loud, unsplit), second heart sounds (normal, reduced intensity, loud, unsplit), fourth heart sounds gallop, fourth heart sound plus first heart sound, hypertrophic cardiomyopathy, mid-systolic click, mitral regurgitation (mild, moderate, severe, pan-systolic murmur), mitral stenosis (mild, moderate, severe, diastolic murmur, regurgitation mild rheumatic), mitral valve leaflet prolapse, mitral valve prolapse (click with late systolic murmur, mild systolic click, standing), myocarditis, opening snap and second heart sound, pleural rubs, mitral prosthetic heart sound, second heart sound and a tumor plop, second heart sound and late systolic click, and/or third heart sound-physiologic, among others. Exemplary patches 12 that may be used for each pathology are shown in Table 4. It should be understood that Table 4 is merely exemplary and more patches may be used for a particular sound, less patches may be used for a particular sound, or different locations may be used for a particular sound.

TABLE 4

| | | |
|---|---|---|
| | Front Patch | |
| Type of Pathology | Placement | Back Patch Placement |

| | |
|---|---|
| Arrhythmias | #1, #2, #3, #4, #5 |
| Heart Murmur | #1, #2, #3, #4, #5 |
| Abnormal Splitting | #1, #2, #3, #4, #5 |
| Heart Gallop | #1, #2, #3, #4, #5 |
| Heart Clicks | #1, #2, #3, #4, #5 |
| Pericardial Rub | #1, #2, #3, #4, #5 |
| Muffled Heart Sounds | #1, #2, #3, #4, #5 |
| Ascending Aortic | #1, #2, #3, #4, #5 |
| Aneurysm | |
| Aortic Stenosis | #3, #4, #5 |
| Aortic, Mitral, Tricuspid | #1, #2, #3, #4, #5 |
| Valve Regurgitation | |
| Carotid Assessments | #6, #7 |
| Jugular Vein Distention | #6, #7 |
| (JVD) Assessments | |
| Ankle Brachial Index (ABI) | #8, #9, #14, #15 |
| Assessments | |
| Arteriovenous Fistula | #6, #7, #8, #9, #10, |
| (AVF) Assessments | #11, #12, #13 |
| Arteriovenous Graft (AVG) | #6, #7, #8, #9, #10, |
| Assessments | #11, #12, #13 |
| Aortic Prosthetic Heart | #1, #2, #3, #4, #5 |
| Sound Aortic Ejection | |
| Click | |
| Aortic Sclerosis | #1, #2, #3, #4, #5 |
| Aortic Stenosis | #1, #2, #3, #4, #5 |
| Aortic Regurgitation | #1, #2, #3, #4, #5 |
| Aortic Coarctation | #1, #2, #3, #4, #5 |
| Aortic Ejection Click - First | #1, #2, #3, #4, #5 |
| Heart Sound plus Aortic | |
| Ejection Click | |
| Aortic Prosthetic Heart | #1, #2, #3, #4, #5 |
| Sound | |
| Aortic Intensity - Second | #1, #2, #3, #4, #5 |
| Heart Sound | |
| Atrial Septal Defect | #1, #2, #3, #4, #5 |
| Innocent Murmur | #1, #2, #3, #4, #5 |
| Patent Ductus Arteriosus | #1, #2, #3, #4 |
| Pulmonary Stenosis | #1, #2, #3, #4, #5 |
| Pulmonic Regurgitation | #1, #2, #3, #4, #5 |
| Second Heart Sound | #1, #2, #3, #4, #5 |
| Acute Pericarditis | #1, #2, #3, #4, #5 |
| Mediastinal Crunch | #1, #2, #3, #4 |
| Arrhythmogenic Right | #1, #2, #3, #4, #5 |
| Ventricular Dysplasia | |
| Ebstein's Anomaly | #1, #2, #3, #4, #5 |
| First Heart Sound | #1, #2, #3, #4, #5 |
| Innocent Systolic Ejection | #1, #2, #3, #4, #5 |
| Murmur | |
| Tetralogy of Fallot | #1, #2, #3, #4, #5 |
| Tricuspid Regurgitation | #1, #2, #3, #4, #5 |
| Ventricular Septal Defect | #1, #2, #3, #4, #5 |
| Commotio Cordis | #1, #2, #3, #4, #5 |
| First Heart Sounds | #1, #2, #3, #4, #5 |
| Second Heart Sounds | #1, #2, #3, #4, #5 |
| Fourth Heart Sounds | #1, #2, #3, #4, #5 |
| Gallop | |
| Fourth Heart Sound Plus | #1, #2, #3, #4, #5 |
| First Heart Sound | |
| Hypertrophic | #1, #2, #3, #4, #5 |
| Cardiomyopathy | |
| Mid-Systolic Click | #1, #2, #3, #4, #5 |
| Mitral Regurgitation | #1, #2, #3, #4, #5 |
| Mitral Stenosis | #1, #2, #3, #4, #5 |
| Mitral Valve Leaflet | #1, #2, #3, #4, #5 |
| Prolapse | |
| Mitral Valve Prolapse | #1, #2, #3, #4, #5 |
| Myocarditis | #1, #2, #3, #4, #5 |
| Opening Snap and | #1, #2, #3, #4, #5 |
| Second Heart Sound | |
| Pleural Rubs | #1, #2, #3, #4, #5 |
| Mitral Prosthetic Heart | #1, #2, #3, #4, #5 |
| Sound | |
| Second Heart Sound and | #1, #2, #3, #4, #5 |
| a Tumor Plop | |
| Second Heart Sound and | #1, #2, #3, #4, #5 |
| Late Systolic Click | |
| Third Heart Sound - | #1, #2, #3, #4, #5 |
| Physiologic | |

More than one type of cardiovascular sound and/or pathology may be monitored, analyzed, or detected at any one time.

In some embodiments, at least one of the plurality of patches 12 is used to monitor gastrointestinal sounds. The gastrointestinal sounds may comprise normal peristalsis GI sounds, hypoactive peristalsis GI sounds, hyperactive peristalsis GI sounds, stomach sounds, small intestine sounds, and/or large intestine sounds, among other gastrointestinal sounds.

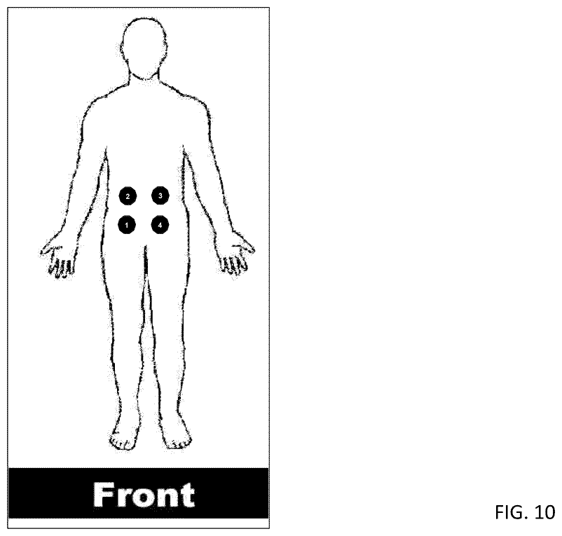

The gastrointestinal sound telemetry that is continuously captured includes, but is not limited to, a pitch of the sound, a frequency of the sound, a duration of the sound (i.e., how long the sound lasted), an amplitude of the sound, a height of the sound, an intensity of the sound, a timing of the sound (i.e., the time at which the sound occurred), a tone of the sound (i.e., a sound quality), and/or a harmonic of the sound, among others. To analyze specific gastrointestinal sounds, particular observation locations for the plurality of patches 12 are chosen. Exemplary observation locations for gastrointestinal sounds are shown in FIG. 10 for the front and the back of the patient. Exemplary patches 12 that may be used for each type of gastrointestinal sound are shown in Table 5. It should be understood that Table 5 is merely exemplary and more patches may be used for a particular sound, less patches may be used for a particular sound, or different locations may be used for a particular sound.

TABLE 5

| | | |
|---|---|---|
| Type of | | |
| Gastrointestinal Sound | Front Patch Placement | Back Patch Placement |

| | |
|---|---|
| Normal Peristalsis GI | #1, #2, #3, #4 |
| Sounds | |
| Hypoactive Peristalsis | #1, #2, #3, #4 |
| GI Sounds | |
| Hyperactive Peristalsis | #1, #2, #3, #4 |
| GI Sounds | |
| Stomach Sounds | #1, #2 |
| Small Intestine Sounds | #1, #2, #3, #4 |
| Large Intestine Sounds | #1, #2, #3, #4 |

Pathologies or anomalies that may be monitored for and/or detected using gastrointestinal sounds include, but are not limited to, borborygmus sound assessments, hyperactive/hypoactive bowel sounds, Crohn's disease, irritable bowel syndrome, bowel obstructions, gastroenteritis, intestinal obstruction, and/or colonic volvulus, among others. Exemplary patches 12 that may be used for each pathology are shown in Table 6. It should be understood that Table 6 is merely exemplary and more patches may be used for a particular sound, less patches may be used for a particular sound, or different locations may be used for a particular sound.

TABLE 6

| | | |
|---|---|---|
| Type of Pathology | Front Patch Placement | Back Patch Placement |

| | |
|---|---|
| Borborygmus Sound | #1, #2, #3, #4 |
| Assessments | |
| Hyperactive/Hypoactive | #1, #2, #3, #4 |
| bowel sounds | |
| Crohn's Disease | #1, #2, #3, #4 |
| Irritable Bowel | #1, #2, #3, #4 |
| Syndrome | |
| Bowel Obstructions | #1, #2, #3, #4 |
| Gastroenteritis | #1, #2, #3, #4 |
| Intestinal Obstruction | #1, #2, #3, #4 |
| Colonic Volvulus | #1, #2, #3, #4 |

More than one type of gastrointestinal sound and/or pathology may be monitored, analyzed, or detected at any one time.

In some embodiments, at least one of the plurality of patches 12 is used to monitor body temperature, axillary temperature, forehead temperature, temporal artery temperature, and/or serum pressure oxygen saturation (SpO2), among other parameters.

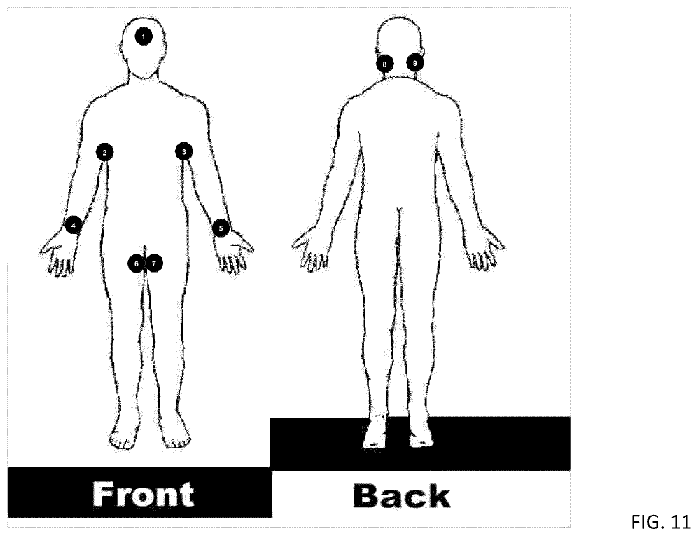

To analyze these parameters, particular observation locations for the plurality of patches 12 are chosen. Exemplary observation locations are shown in FIG. 11 for the front and the back of the patient. Exemplary patches 12 that may be used for each type of parameter are shown in Table 7. It should be understood that Table 7 is merely exemplary and more patches may be used for a particular parameter, less patches may be used for a particular parameter, or different locations may be used for a particular parameter.

TABLE 7

| Type of Physiological | | |
|---|---|---|
| Signal | Front Patch Placement | Back Patch Placement |
| Body Temperature | #1, #2, #3, #4, #5, #6, | #8, #9 |
| | #7 | |
| Axillary Temperature | #1, #2, #3, #4, #5, #6, | #8, #9 |
| | #7 | |
| Forehead Temperature | #1 | |
| Temporal Artery | #1, #2, #3, #4, #5, #6, | #8, #9 |
| Temperature | #7 | |
| Serum Pressure Oxygen | #1, #2, #3, #4, #5, #6, | #8, #9 |
| Saturation | #7 | |

Pathologies or anomalies that may be monitored for and/or detected using temperature and/or pressure oxygen saturation include, but are not limited to, anomalous body temperature and/or anomalous serum pressure oxygen saturation, among others. Exemplary patches 12 that may be used for each pathology are shown in Table 8. It should be understood that Table 8 is merely exemplary and more patches may be used for a particular parameter, less patches may be used for a particular parameter, or different locations may be used for a particular parameter.

TABLE 8

| Type of Pathology | Front Patch Placement | Back Patch Placement |
|---|---|---|
| Anomalous Body | #1, #2, #3, #4, #5, #6, | #8, #9 |
| Temperature | #7 | |
| Anomalous Serum | #1, #2, #3, #4, #5, #6, | #8, #9 |
| Pressure Oxygen | #7 | |
| Saturation | | |

More than one of temperature and pressure oxygen saturation (and their corresponding anomalies) may be monitored, analyzed, or detected at any one time. More than one physiological signal (i.e., respiratory sounds, cardiovascular sounds, gastrointestinal sounds, temperature, and/or pressure oxygen saturation) may be monitored, analyzed, or detected at any one time. For example, one set of patches 12 may be configured to detect, measure, or monitor stomach sounds, while another set of patches 12 may be configured to detect, measure, or monitor body temperature.

In some embodiments, the plurality of patches 12 may be used for an adult or a pediatric patient. In some embodiments, the plurality of patches 12 are different for an adult and a pediatric patient. For example, the size of the patches 12 may be adjusted as between an adult and a pediatric patient.

While the disclosure has been illustrated and described in detail in the foregoing drawings and description, the same is to be considered as exemplary and not restrictive in character, it being understood that only illustrative embodiments thereof have been shown and described and that all changes and modifications that come within the spirit of the disclosure are desired to be protected.

What is claimed is:

1. An electronic stethoscope assembly comprising:
a plurality of patches configured to be adhered to a patient, each of the plurality of patches including at least one sensor configured to measure physiological signals upon placement in contact with the patient and a transceiver configured to transmit physiological signal data, the plurality of patches including a command patch and at least one follower patch in communication with the command patch to transmit the physiological signal data measured by the at least one follower patch to the command patch, and
a controller in communication with the command patch to receive a set of physiological signal data from the command patch for processing and analyzing of the set of physiological signal data so that the set of physiological signal data can be continuously collected and monitored to identify a pathology present in the set of physiological signal data, the set of physiological signal data including the physiological signal data measured by the command patch and the physiological signal data measured by the at least one follower patch.

2. The assembly of claim 1, wherein the controller is in communication with a user interface and the controller is configured to output the set of physiological signal data to the user interface.

3. The assembly of claim 2, wherein a respective location of each of the plurality of patches on the patient is displayed on the user interface, and in response to identifying the presence of the pathology in the set of physiological signal data, the respective patch of the plurality of patches that measured the respective physiological signal data included in the set of physiological signal data having the pathology is illuminated on the user interface.

4. The assembly of claim 1, wherein the physiological signal data measured by the command patch is filtered with a first bandpass filter and the physiological signal data measured by the at least one follower patch is filtered with a second bandpass filter different than the first bandpass filter so that the physiological signal data measured by the command patch is related to a different physiological signal than the physiological signal data measured by the at least one follower patch.

5. The assembly of claim 1, wherein the at least one sensor comprises a transducer.

6. The assembly of claim 1, wherein the at least one sensor comprises a temperature sensor.

7. The assembly of claim 1, wherein the at least one sensor comprises a photoplethysmogram sensor.

8. The assembly of claim 1, wherein the at least one sensor of the command patch consists of a transducer, and the at least one sensor of the follower patch comprises one of a temperature sensor and a photoplethysmogram sensor.

9. The assembly of claim 1, wherein each of the plurality of patches comprises an adhesive surface configured to adhere to the patient.

10. The assembly of claim 1, wherein the command patch determines a location of the at least one follower patch on the patient based, at least in part, on an angle of arrival of the physiological signal data from the at least one follower patch.

11. A method comprising:
coupling a plurality of patches to a patient, the plurality of patches including a command patch and at least one follower patch,
measuring, via at least one sensor of each of the plurality of patches, physiological signals of the patient,
transmitting physiological signal data from the at least one follower patch to the command patch,
transmitting a set of physiological signal data from the command patch to a controller, the set of physiological signal data including physiological signal data measured by the command patch and the physiological signal data measured by the at least one follower patch, and
processing, via the controller, the set of physiological signal data so that the set of physiological signal data can be continuously collected and monitored to identify a pathology present in the set of physiological signal data.

12. The method of claim 11, further comprising outputting the set of physiological signal data to a user interface.

13. The method of claim 12, wherein a respective location of each of the plurality of patches on the patient is displayed on the user interface, and the method further comprises, in response to identifying the presence of the pathology in the set of physiological signal data, illuminating the respective patch of the plurality of patches that measured the respective physiological signal data included in the set of physiological signal data having the pathology on the user interface.

14. The method of claim 11, wherein the step of processing includes filtering the physiological signal data measured by the command patch with a first bandpass filter and filtering the physiological signal data measured by the at least one follower patch with a second bandpass filter different than the first bandpass filter so that the physiological signal data measured by the command patch is related to a different physiological signal than the physiological signal data measured by the at least one follower patch.

15. The method of claim 11, wherein the at least one sensor comprises a transducer.

16. The method of claim 11, wherein the at least one sensor comprises a temperature sensor.

17. The method of claim 11, wherein the at least one sensor comprises a photoplethysmogram sensor.

18. The method of claim 11, wherein the at least one sensor of the command patch consists of a transducer, and the at least one sensor of the follower patch comprises one of a temperature sensor and a photoplethysmogram sensor.

19. The method of claim 11, wherein the step of coupling includes adhering an adhesive surface of each of the plurality of patches to the patient.

20. The method of claim 11, further comprising determining, via the command patch, a location of the at least one follower patch on the patient based, at least in part, on an angle of arrival of the physiological signal data from the at least one follower patch.
